(19) United States
(12) Patent Application Publication
(10) Pub. No.: US 2007/0122862 A1
(43) Pub. Date: May 31, 2007

(54) Screening for lipolytic enzymes or amidase activity

(75) Inventors: Kim Borch, Birkerod (DK); Christel Thea Jorgensen, Lyngby (DK)

Correspondence Address: NOVOZYMES NORTH AMERICA, INC., 500 FIFTH AVENUE, SUITE 1600, NEW YORK, NY, 10110, US

(73) Assignee: NOVOZYMES A/S, Bagsvaerd (DK)

(21) Appl. No.: 10/576,497

(22) PCT Filed: Oct. 29, 2004
(86) PCT No.: PCT/DK04/00748

§ 371 Date: Apr. 18, 2006

(30) Foreign Application Priority Data
Oct. 29, 2003 (DK) PA 2003 01596

(51) Int. Cl. C12Q 1/34

(52) U.S. Cl. 435/18

(57) ABSTRACT

A method of testing samples for their enzymatic activity for hydrolysis of a particular ester or amide bond in a substrate uses a test substrate with one or more polyunsaturated fatty acyl groups linked through amide or ester bonds. The release of the polyunsaturated fatty acid is detected by the use of a lipoxygenase to convert the polyunsaturated fatty acid into a hy-droperoxide which is then detected, e.g. through a color reaction.

### FIELD OF THE INVENTION

The present invention relates to a method for detecting ½ hydrolytic activity towards a particular ester or amide bond in a substrate.

### BACKGROUND OF THE INVENTION

Lipolytic enzymes such as triacyl glycerol lipase, phospholipases, and galactolipase are used industrially, e.g. in baking as additives to dough, and in detergents. In the development of lipolytic enzymes for baking it is of interest to test candidate enzymes for their hydrolytic activity on ester bonds in various substrates such as triacyl glycerol, phospholipids and galactolipids (WO 0032758).

Amidases can be used industrially, e.g. in the hydrolysis of nylon.

Lipolytic enzyme or amidase activity in a sample is conventionally detected by incubating the sample with a lipid or amide and detecting the formation of free non-esterified fatty acid. The formation of fatty acid may be followed by titration or by enzymatic colorimetric methodology.

U.S. Pat. No. 4,301,244 discloses such a method which relies upon the acylation of coenzyme A(CoA) by the fatty acids in the presence of added acyl-CoA synthetase (ACS). The acyl-CoA produced is oxidized by added acyl-CoA oxidase (ACOD) with the generation of hydrogen peroxide. Hydrogen peroxide, in the presence of peroxidase (POD) permits the oxidative condensation of 3-methyl-N-ethyl-N-(b-hydroxyethyl)-aniline (MEHA) with 4-aminoantipyrine to form a purple color which can be measured spectrophotometrically at 550 nm.

CA 1120833 and H. F. Proelss and B. W. Wright, Clin. Chem., 23 (3), 522-531 (1977) disclose a test for lipase activity in a biological fluid, using trilinolein as a substrate.

S. P. Wolff, Methods in Enzymology, vol. 223, pages 182-189. (1994) is titled “Ferrous ion oxidation in presence of ferric ion indicator xylenol orange for measurement of hydroperoxides”.

### SUMMARY OF THE INVENTION

The inventors have developed a method of testing samples for their enzymatic activity for hydrolysis of a particular ester or amide bond in a substrate. The method uses a test substrate with one or more polyunsaturated fatty acyl groups linked through amide or ester bonds. The release of the polyunsaturated fatty acid is detected by the use of a lipoxygenase to convert the polyunsaturated fatty acid into a hydroperoxide which is then detected, e.g. through a color reaction.

The method can be used to test for a particular enzymatic activity with a substrate specificity of interest. Thus, by a suitable choice of test substrate, the method can be used to detect various specificities of amidase or lipolytic enzyme activities, i.e. enzyme activities classified in EC 3.5.1 and 3.1.1.

Accordingly, the lipolytic enzyme or amidase activity in a sample may be detected by a method, comprising the steps of:

a) incubating the sample with a substrate having one or two polyunsaturated fatty acyl groups linked through amide or ester bonds,

b) simultaneously or subsequently incubating the sample with a lipoxygenase to allow formation of a hydroperoxide of the polyunsaturated acid, and

c) detecting the formation of the hydroperoxide.

Further, lipolytic enzyme or amidase activity in a test sample may be detected by a method, comprising the sequential steps of:

a) incubating the sample with a lipoxygenase and a substrate having one or more polyunsaturated fatty acyl groups linked through amide or ester bonds, to allow formation of a hydroperoxide of the polyunsaturated acid,

b) incubating with a ferrous salt and xylenol orange to allow color generation, and

c) detecting color generation.

### DETAILED DESCRIPTION OF THE INVENTION

Test Substrate

The substrate is an ester or amide of the general formula (A-CO—X)ₙB wherein A-CO is polyunsaturated fatty acyl, X is O (oxygen) or NH, n is an integer (particularly 1 or 2), and B is an organic group. The substrate is hydrolyzed into free polyunsaturated fatty acid A-COOH and a hydroxyl compound (alcohol or phenol) or amine (A-CO—X)ₙ₋₁B—XH or B(XH)ₙ. To make the method more specific, the substrate may have a single polyunsaturated fatty acyl group (n=1) or two such groups (n=2) arranged symmetrically.

The poly-unsaturated fatty acyl group and the corresponding poly-unsaturated fatty acid may contain a cis,cis-1,4-pentadiene unit, such as linoleoyl and linoleic acid (18 carbon atoms, 2 double bonds), linolenoyl and linolenic acid (18:3), arachidonoyl and arachidonic acid (20:4), eicosapentaenoyl and eicosapentaenoic acid (EPA, 20:5) and/or docosahexaenoyl and docosahexaenoic acid (DHA, 22:6).

The substrate may be a lipid having one or more (particularly one or two) polyunsaturated fatty acyl groups linked through amide or ester bonds. The lipid may in particularly be a polar lipid such as a phospholipid, a lysopholipid or a galactolipid. The substrate may be isolated from natural sources or may be commercially available. The isolated substrate may contain a mixture of polyunsaturated fatty acyl groups together with other acyl groups.

Some examples are:
Phospholipids, e.g. phosphatidyl inositol (PI), phosphatidyl ethanolamine (PE), phosphatidyl choline (PC), N-acyl phosphatidyl ethanolamine (APE)
Lysophospholipids, e.g. lyso-phosphatidyl choline (LPC), lyso-phosphatidyl ethanolamine (LPE), N-acyl lysophosphatidyl ethanolamine (ALPE)
Galactolipids, e.g. digalactosyl diglyceride (DGDG), monogalactosyl diglyceride (MGDG), digalactosyl monoglyceride (DGMG)
Glycerides (triglycerides (TG), diglycerides (DG), monoglycerides (MG)) such as di- or mono-linolein
Wax-esters

Further, the substrate may be an ester prepared synthetically, e.g. by attaching a polyunsaturated fatty acyl group (such as linoleoyl) to a hydroxyl group of the following compounds:
Aliphatic alcohols (primary, secondary, tertiary, e.g. 1,2-di-O-butyl-glycerol and 1,3-di-O-butylglycerol)
Amino acid derivatives (e.g. Ser, Thr, Tyr)
Galactolipids, e.g. digalactosyl diglyceride (DGDG), monogalactosyl diglyceride (MGDG), digalactosyl monoglyceride (DGMG)
Peptides (oligo or poly containing a hydroxyl-amino acid, Ser, Thr or Tyr)
Saccharides (mono/oligo/poly, e.g. glucose, sucrose, starch)
Alkyl and aryl glycosides (e.g. ethyl α,β-glucoside)
Polyols (e.g. glycerol, sorbitol, ethylene glycol)
Glycerides (e.g. diglycerides (DG), monoglycerides (MG))
Sterols (e.g. cholesterol, sitosterol)
Glycolipids (e.g. steryl glycosides, gangliosides, cerebrosides)
Phenolic compounds, e.g. phenyl or p-nitrophenyl linoleate

Finally, the substrate may be an amide prepared synthetically, e.g. by attaching a polyunsaturated fatty acyl group (such as linoleoyl) to an amino group of the following amines:
Amino sugars (e.g. glucosamine)
Phosphatidylethanolamines (e.g. PE)
Aliphatic or aromatic amines (e.g. 1,6-diaminohexane)
Amino acid derivatives and peptides
Ceramides
   Lipoxygenase

The method uses a lipoxygenase, preferably with a high activity for free polyunsaturated acid and a low activity for the polyunsaturated fatty acyl group in the substrate.

The lipoxygenase (EC 1.13.11.12) is an enzyme that catalyzes the oxygenation of poly-unsaturated fatty acids such as linoleic acid, linolenic acid and arachidonic acid, which contain a cis,cis-1,4-pentadiene unit and produces hydroperoxides of these fatty acids. The lipoxygenase is able to oxidize substrates containing a cis-cis-pentadienyl moiety. The lipoxygenase may be a 9-lipoxygenase with the ability to oxidize the double bond between carbon atoms 9 and 10 in linoleic acid and linolenic acid, or it may be a 13-lipoxygenase with the ability to oxidize the double bond between carbon atoms 12 and 13 in linoleic acid and linolenic acid.

The lipoxygenase may be from animal, plant or microbial source. A plant lipoxygenase may be from plants of the pulse family (Fabaceae), soybean (lipoxygenases 1, 2 and 3), cucumber, or barley. A microbial lipoxygenase may be from a yeast such as Saccharomyces cerevisiae, a thermophilic actinomycete such as Thermoactinomyces vulgaris or Thermomyces, e.g. T. lanuginosus, or from fungi.

A fungal lipoxygenase may be derived from Ascomycota, particularly Ascomycota incertae sedis e.g. Magnaporthaceae, such as Gaeumannomyces or Magnaporthe, or anamorphic Magnaporthaceae such as Pyticulaia, or alternatively anamorphic Ascomycota such as Geotrichum, e.g. G. candidum. The fungal lipoxygenase may be from Gaeummanomyces graminis, e.g. G. graminis var. graminis, G. graminis var. avenae or G. graminis var. tritici, (WO 0220730) or Magnaporthe salvinii (WO 2002086114). Also, a fungal lipoxygenase may be from Fusadium such as F. oxysporum or F. proliferatum, or Penicillium sp.

Test Samples

The method can be applied to any kind of samples, crude or purified, e.g. soil samples, isolated microbial strain (e.g. cultivated on an appropriate medium), or enzymes in crude or purified form. The enzymes may be isolated from nature or may be variants formed by modifying the amino acid sequence of a parent lipolytic enzyme or amidase.

Screening Method

The screening method can be carried out in a cuvette, or it can be used for high-throughput screening in a microtiter plate.

Particularly in screening for detergent enzymes, the substrate may be applied to a textile swatch which is then treated in a detergent solution with a lipolytic enzyme to be tested and a lipoxygenase. As an example, a solution of trilinolein (e.g. 25% by weight) in n-hexane or n-heptane may be applied to small pieces of textile from which the solvent is evaporated. The textile pieces may be fitted into the holes of a microtiter plate, with 5 micro-l of trilinolein solution applied to each textile piece.

Detection of Hydroperoxide

The method relies on detection of a hydroperoxide formed by the action of the lipoxygenase. The detection can conveniently be done by the color generation with various known reagents such as xylenol orange or diphenyl-1-pyrenylphosphine (DPPP). Other reagents can be found in Chapter 19 of Handbook of Fluorescent Probes and Research Products, 9th Editon, published by Molecular Probes.

Enzymatic Activity

Depending on the choice of the amide or ester substrate, the method can be used to detect an amidase (EC 3.5.1) or a lipolytic enzyme (EC 3.1.1) with a particular substrate specificity. Thus, the substrate can be chosen so as to detect any of the following enzyme activities:

EC 3.1.1.1 carboxylesterase

EC 3.1.1.2 arylesterase

EC 3.1.1.3 triacylglycerol lipase

EC 3.1.1.4 phospholipase A₂

EC 3.1.1.5 lysophospholipase

EC 3.1.1.6 acetylesterase

EC 3.1.1.7 acetylchoiinesterase'

EC 3.1.1.8 cholinesterase

EC 3.1.1.13 sterol esterase

EC 3.1.1.26 galactolipase

EC 3.1.1.32 phospholipase A₁

EC 3.1.1.50 wax-ester hydrolase

EC 3.5.1.13 aryl-acylamidase

EC 3.5.1.14 aminoacylase

EC 3.5.1.15 aspartoacylase

EC 3.5.1.17 acyl-lysine deacylase

Use of Detected Enzyme

The method can be used to select enzymes for various uses by a suitable choice of the test substrate.

Thus, a wheat lipid can be used to select a lipolytic enzyme for use addition to a dough in the preparation of baked products.

An aliphatic amine (e.g. 1,6-diaminohexane) can be used to select an amidase for use in the hydrolysis of nylon.

A substrate applied to textile can be used to screen for lipolytic enzymes for use in detergents.

### EXAMPLES

Methods

Synthesis of Linoleoyl Esters of Monohydroxy Compounds, General Procedure

The alcohols were converted into the linoleic acid ester by standard esterification procedures in an organic solvent (typically dry dichloromethane or pyridine) using 1.2 eq. (molar basis) of linoleoyl chloride or linoleoyl anhydride in the presence of 0.1 eq. DMAP (N,N-dimethylaminopyridine) and 1.2 eq. of base (pyridine or triethylamine). The acid chloride/anhydride was added to a solution of the other compounds at 0° C. under nitrogen. After stirring overnight (N₂) the mixture was filtered, extracted twice with sat. NaHCO₃ and then extracted with water. Drying (MgSO₄ or Na₂SO₄) and concentration afforded an oil that was normally purified by flash chromatography. Eluents used were typically mixtures of heptane/ethylacetate. Structures were confirmed by ¹H NMR spectroscopy.

For enantiopure alcohols or amines containing base sensitive chiral centers, the esterification can also be achieved using linoleic acid and DCC (dicyclohexylcarbodiimide).

Monoacylation of Polyhydroxy Compounds, General Procedure

The polyol, typically carbohydrates (mono, di or oligosaccharides), was esterified with linoleic acid or linoleic acid methyl ester using immobilized lipase B from Candida antarctica (WO 8802775) Novozyme 435 in organic solvent or without solvent. This was done in analogy with published procedures: Adelhorst, K.; Björkling, F.; Godtfredsen, S. E.; Kirk, O., Synthesis, 1990, 112-115. Mutua, L; Akoh, C. C.; J. Am. Oil Chem. Soc. 70, 1, 4346 (1993). Anderson, E. M.; Larsson, K. M.; Kirk, O.; Biocatalysis and Biotransformation, 16, 181-204 (1998).

Synthesis of Linoleoyl Amides, General Procedure

The linoleoyl amides were prepared analogous to the linoleoyl esters except that no DMAP were used and TEA (triethylamine) or DIPEA (diisopropyethylamine) was used as base.

Screening Method

The substrate is added to a concentration of 0.44 mg/ml and a total volume of 60 microliter in a buffer at pH 7.0 containing 5 mM CaCl₂, 50 mM HEPES, 50 mM Borate and 50 mM Actetic acid and homogenized for 1 minute by sonication at 60° C. Upon cooling to room temperature (25° C.) lipoxygenase (e.g. from Magnaporthe salvinii) is added to a final concentration corresponding to approximately 0.02 mg/ml (total volume 80 microliter). 20 microliter of the test sample is added to an enzyme concentration of approximately 0.002 mg/ml as enzyme protein, and the reaction mixture is incubated (A).

After 30 minutes, 20 microliter of the reaction mixture is added into 180 microliter of a solution with the following composition*:
100 microliter 0.01 M Xylenol Orange in Methanol
100 microliter 2.5 M H₂SO₄
100 microliter 0.025 M Fe(NH₄)₂(SO₄)₂.6H₂O
100 microliter 0.4 M Butylated Hydroxytoluene in Methanol.
8.8 ml Methanol
800 microliter desalted water

The reaction mixture (200 microliter) is incubated (B) for 60 minutes at 25° C. and OD560 is determined. Reaction runs in 96-well microtiterplate format and lipase-reaction is quantifyied upon determination of OD560 in triplicate, and upon substraction of similar blank experiments without lipase in incubation A. In blank experiment the sample is added in incubation B where pH<2 and the lipolytic enzyme activity is normally insignificant.

### Example 1

### Isolation of Flour Lipids MGDG, DGDG, APE and ALPE

Wheat flour (1 kg) was extracted twice with MeOH (1.5 L, stirring for 30 min). The extracts were concentrated and the residue re-dissolved in hexane (1 L) and concentrated. Yield of lipid extract: 8.5 g. The lipid extract was applied to a column packed with silica gel (120 g), which was preconditioned with 1 L of hexane/2-propanol/butanol/H₂O (60:30:7:3). Neutral lipids and carotenoids were removed by eluation with hexane (800 mL) and then EtOAc (1.2 L). Galactolipids were removed by eluting with with toluene/acetone (1:1, 800 mL, MGDG) and acetone (9 L, DGDG). Finally, phospholipids (˜1.1 g) could be eluated with MeOH (1 L). The individual phospholipids could be isolated by flash chromatography (CHCl₃/MeOH/H₂O: 65:25:4) to give pure fractions of APE and ALPE. The structures were verified by ¹H NMR and MS analysis.

### Example 2

### Isolation of Polar Lipid Mixture

A mixture of polar lipids (DGDG, MGDG, APE, ALPE) was isolated from wheat flour as follows.

Wheat flour (1.5 kg) was stirred in a beaker with MeOH (2.25 L) using a mechanical stirrer (350 rpm). After 20 min the thick suspension was filtered on a G1 filter (27×22 cm). The wetted flour was re-suspended and stirred with an additional amount of MeOH (2 L) and filtered again. The pooled MeOH phases were concentrated on a rotary evaporator and the residue was dissolved in hexane (1 L). Filtration and concentration to dryness left 22.6 g of lipid extract (this yield may vary). This extract contained both polar and non-polar lipids.

A silica gel column was packed using 270 g of Merck silica gel 60 (270 g) and an eluent of hexane/2-propanol/1-butanol/water (600:300:70:30). The extracted lipids was then dissolved in a small volume of the eluent and applied to the column. The column was eluted with first hexane (1400 mL), next ethyl acetate (2100 mL) and finally MeOH (2800 mL). The MeOH fraction was concentrated (careful, may sputter) to give 4.9 g of polar lipid extract. Storage: freezer, over nitrogen If possible.

### Example 3

### Preparation of (+/−) 3-O-Linoleoyl-1,2-di-O-butyl glycerol

The alcohol 1,2-di-O-butyl glycerol was prepared as described in Ciuffreda, P.; Loseta, A.; Manzocchi, A.; Santaniello, E.; Chem. Phys. Lip.; 111, 105-110 (2001), essentially as follows.

The alcohol (1.6 g, 8.0 mmol) and triethylamine (1.3 mL, 9.5 mmol, 1.2 eq.) are dissolved in dry CH₂Cl₂ (25 mL) and linoleoyl chloride (3.1 mL, 9.5 mmol) and DMAP (0.10 g, 0.80 mmol) is added at 0° C. under nitrogen. After 30 min the solution is allowed to reach room temperature and then stirred overnight (nitrogen). The mixture is filtered and washed with water, diluted NaHCO₃ (aq) and water before being dried (Na₂SO₄) and concentrated.

Yield of crude oily product was 3.3 g. The product was purified by flash chromatography (EtOAc/heptane 1:15) to give 1.4 g (50%) of the title compound as an oily product.

¹H NMR (CDCl₃): 5.35 ppm (m, C═CH), 4.24 ppm (dd, 1H, H-3a), 4.10 ppm (dd, 1H, H-3b), 3.61 ppm (m, 1H, H-2), 3.55 ppm (t, 2H, CH₂O), 3.45 ppm (m, 4H, CH₂O), 2.78 ppm (t, ═CHCH₂CH═), 2.30 ppm (t, 2H, CH₂COO), 2.02 ppm (m, CH₂CH═), 1.64 ppm (p, 2H, CH₂CH₂COO), 1.54 ppm (p, 4H, CH₂CH₂O), 1.36 ppm (m, 4H, CH₂), 1.31 ppm (m, CH₂), ˜0.90 ppm (3 x t, 9H, CH₃).

### Example 4

### Activity of Lipolytic Enzymes on Ester Substrates

The following substrates were prepared, and various lipolytic enzymes were tested with each substrate:
Galactolipid: Digalactosyl diglyceride (DGDG) and monogalactosyl diglyceride (MGDG)
Phospholipid: Lecithin
Sterol ester: Cholesterol linoleate
Wax ester: Arachidyl linoleate
2-position of glycerides: 1,3-dibutyl-2-linoleyl glycerol
Glycerides: Trilinolein
Linoleic acid Isopropyl ester
Linoleic acid Syringaldazine (4-Hydroxy-3,5-dimethoxybenzaldehyde azine) diester (poor solubility)
Linoleic acid Phenyl ester
Soy bean oil (with a content of linoleic acid, mainly in the 2-position)
Substrates for testing positional specificity of lipases: 1,3-Dibutyl-2-Linoleoyl-Glycerol; 2,3-Dibutyl-1-Linoleoyl-Glycerol
1,6-Diaminohexane Linoleic Acid diamide (poor solubility), tested in the presence of a surfactant
Substrates for testing phospholipase specificity: L-a-Phosphatidylcholine; Dilinoleoyl-Phosphatidylcholine
Ethyl-6-O-Linoleoyl-alfa/beta-glycoside
Ferulic acid linoleate
Serine linoleate
Dilinolein

With each substrate, the positive or negative results for the various enzymes confirmed previous knowledge of the enzyme's substrate specificity.

### Example 5

### Comparison with Plate Assay

Five variants of a parent lipolytic enzyme were prepared by amino acid modification and were tested in lipid hydrolysis for 30 minutes at 25° C. with MGDG or APE as substrate at 1.5 mM using lipolytic enzyme A280=0.04. For comparison, lipid hydrolysis was also tested in a plate assay with APE/ALPE. Results are given as 0 or on a scale from * (very low activity) to ***** (very high activity).

| | |
|---|---|
| Invention | Comparison |

| | MGDG | APE | APE/ALPE |
|---|---|---|---|
| Variant 1 | * | 0 | 0 |
| Variant 2 | * | * | 0 |
| Variant 3 | **** | ***** | ***** |
| Variant 4 | ***** | **** | ***** |
| Variant 5 | **** | ***** | ***** |

The results show that the activity towards APE by the method of the invention correlates with the activity by the plate assay.

1. A method for detecting lipolytic enzyme or amidase activity in a sample, comprising the steps of:
a) incubating the sample with a substrate having one or two polyunsaturated fatty acyl groups linked through amide or ester bond(s) to allow hydrolysis of the amide or ester bond(s),
b) simultaneously or subsequently incubating the sample with a lipoxygenase to allow formation of a hydroperoxide of the polyunsaturated acid, and
c) detecting the formation of the hydroperoxide.

2. The method of claim 1 wherein the polyunsaturated fatty acyl group is linoleoyl (18:2).

3. The method of claim 1 wherein the substrate is a polar lipid.

4. The method of claim 3 wherein the substrate is a galactolipid, particularly digalactosyl diglyceride (DGDG) or monogalactosyl diglyceride (MGDG).

5. The method of claim 3 wherein the substrate is a phospholipid, particularly lecithin, L-a-phosphatidylcholine; dilinoleoyl-phosphatidylcholine.

6. The method of claim 1 wherein the substrate is a sterol ester, particularly cholesterol linoleate.

7. The method of claim 1 wherein the substrate is a wax ester, particularly arachidyl linoleate

8. The method of claim 1 wherein the substrate is a monoester, particularly 1,3-dibutyl-2-linoleyl glycerol, 2,3-dibutyl-1-linoleoyl-glycerol or linoleic acid isopropyl ester.

9. The method of claim 1 wherein the substrate is an aryl ester, particularly linoleic acid phenyl ester.

10. The method of claim 1 wherein the substrate is a mono- or diamide, particularly 1,6-diaminohexane linoleic acid diamide.

11. A method of detecting lipolytic enzyme or amidase activity in a test sample, comprising the sequential steps of:
a) incubating the sample with a lipoxygenase and a substrate having one or more polyunsaturated fatty acyl groups linked through amide or ester bonds, to allow formation of a hydroperoxide of the polyunsaturated acid,
b) incubating with a ferrous salt and xylenol orange to allow color generation, and
c) detecting color generation.
